(19) United States
(12) Patent Application Publication
(10) Pub. No.: US 2015/0164868 A1
(43) Pub. Date: Jun. 18, 2015

(54) METHODS OF ADMINISTERING RIFAXIMIN WITHOUT PRODUCING ANTIBIOTIC RESISTANCE

(71) Applicant: Salix Pharmaceuticals, Inc., Raleigh, NC (US)

(72) Inventors: Mark Pimentel, Los Angeles, CA (US); Christopher Chang, Los Angeles, CA (US)

(21) Appl. No.: 14/629,873

(22) Filed: Feb. 24, 2015

Related U.S. Application Data
(63) Continuation of application No. PCT/US2013/059415, filed on Sep. 12, 2013.
(60) Provisional application No. 61/700,151, filed on Sep. 12, 2012.

(51) Int. Cl. A61K 31/437; A61K 45/06

(52) U.S. Cl. CPC A61K 31/437; A61K 45/06

(57) ABSTRACT

Methods of administering a composition comprising rifaximin to a subject in need thereof are provided, wherein the methods do not increase development of rifampicin resistance to a Staphylococcus spp. Methods of reducing the development of rifampicin resistance to a Staphylococcus spp. are also provided, comprising administering a composition comprising rifaximin to a subject in need thereof.

### RELATED APPLICATIONS

This application is a continuation of International Application No. PCT/US2013/059415, filed on Sep. 12, 2013, which claims the benefit of U.S. Provisional Application No. 61/700,151, filed on Sep. 12, 2012. The entire contents of each of the aforementioned applications are incorporated herein by reference.

### BACKGROUND

Rifaximin (INN; see The Merck Index, XIII Ed., 8304) is an antibiotic belonging to the rifamycin class of antibiotics, e.g., a pyrido-imidazo rifamycin. Rifaximin exerts its broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

Although rifaximin is not systemically absorbed, is unrelated to most commonly used systemic antibiotics, and clinical resistance is not recognized as a problem with using rifaximin in IBS, there remain some concerns about bacterial resistance. Since rifaximin is a rifamycin derivative and related to rifampin, there has been concern about whether rifaximin may create a rifampin resistant organism. Even if that were the case, the durability of this resistance has not been tested.

Accordingly, there is a need to address the concern that rifaximin may produce Staphylococcal resistance to rifampicin in vivo.

### SUMMARY

Discussed herein are methods of reducing rifampicin-resistant organisms in a subject in need thereof, comprising administering a composition comprising a therapeutically effective amount of rifaximin to the subject. Embodiments are directed to a method of reducing rifampicin-resistant Staphylococcus spp. organisms in a subject in need thereof, comprising administering a composition comprising a therapeutically effective amount of rifaximin to the subject. In some embodiments, administration of the composition results in a reduction in the number of rifampicin-resistant Staphylococcus spp. organisms compared to a pre-treatment baseline number. In some embodiments, the reduction in the number of rifampicin-resistant Staphylococcus spp. relative to baseline is determined by culturing stool samples obtained from the subject prior and subsequent to administration of the composition.

Embodiments also relate to a method of administering a composition comprising rifaximin to a subject, wherein administration of the composition does not increase the number of rifampicin-resistant Staphylococcus spp. in the subject relative to baseline.

In some embodiments, the threshold mean inhibitory concentration of rifampicin for the Staphylococcus spp. organism is less than about 2.5 μg/mL.

In some embodiments, the subject is administered rifaximin at a dose of about 25 mg to about 6000 mg per day.

In some embodiments, the subject is administered rifaximin at a dose of about 50 mg to about 6000 mg per day. In some embodiments, the subject is administered rifaximin at a dose of between about 100 mg and about 6000 mg. In some embodiments, the subject is administered rifaximin at a dose of between about 50 mg and about 2500 mg BID. In some embodiments, the subject is administered rifaximin at a dose of between about 50 mg and about 2000 mg TID. In some embodiments, the subject is administered rifaximin at a dose of about 200 mg TID. In some embodiments, the subject is administered rifaximin at a dose of about 200 mg BID. In some embodiments, the subject is administered rifaximin at a dose of about 200 mg QD.

In some embodiments, the subject is administered rifaximin at a dose of about 550 mg, 600 mg or 1650 mg TID, QD or BID.

In some embodiments, the subject is administered the composition for between about 1 week and about 24 months.

In some embodiments, the subject is administered the composition for about 10 days.

In some embodiments, the subject is administered the composition such that there is a change in small bowel colonizers.

In some embodiments, the subject is administered the composition such that there is a decrease in small bowel colonizers.

In some embodiments, the subject is administered the composition such that there is a reduction in colonic Escherichia coli (E. coli).

In some embodiments, the subject is administered the composition such that there is a reduction in colonic Klebsiella.

In some embodiments, the subject is administered the composition such that there is a reduction in Campylobacter jejuni (C. jejuni). In some embodiments, the subject is suffering from a bowel disease selected from the group of: an inflammatory bowel disease (IBD), hepatic encephalopathy (HE), enteritis, colitis, irritable bowel syndrome (IBS), diarrhea-predominant irritable bowel syndrome (d-IBS), non-constipation-predominant irritable bowel syndrome (non-C IBS), traveler's diarrhea (TD), a Clostridium difficile infection (CDI), diverticular disease, fibromyalgia (FM), chronic fatigue syndrome (CFS), depression, attention deficit/hyperactivity disorder (ADHD), multiple sclerosis (MS), systemic lupus erythematosus (SLE), small intestinal bacterial overgrowth, chronic pancreatitis, and pancreatic insufficiency.

In some embodiments, the inflammatory bowel disease is selected from the group of: Crohn's Disease and ulcerative colitis.

In some embodiments, the subject is suffering from a bowel disease related to bacterial infection.

In some embodiments, the subject is suffering from a bowel disease related to a bacterial infection that has cleared.

In some embodiments, the subject is suffering from a bowel disease related to a change in intestinal flora.

In some embodiments, the subject is suffering from a change in small bowel flora.

In some embodiments, the subject is suffering from a bowel disease related to an E. coli infection.

In some embodiments, the subject is suffering from a bowel disease related to a Campylobacter jejuni (C. jejuni) infection. In some embodiments, the subject has recovered from a C. jejuni infection.

In some embodiments, the subject is suffering from a bowel disease related to a Klebsiella infection.

In some embodiments, the subject is suffering from a bowel disease related to an Aeromonas infection.

In some embodiments, the subject has excessive flora in their small intestine.

In some embodiments, the subject has increased coliform count in their small bowel.

In some embodiments, the subject is suffering from IBS.

In some embodiments, the subject is suffering from small intestinal bacterial overgrowth.

In some embodiments, the subject is suffering from IBS and small intestinal bacterial overgrowth.

In some embodiments, the subject is suffering from IBS and has increased levels of E. coli, as compared to subjects without IBS.

In some embodiments, the subject is suffering from IBS and has increased levels of Klebsiella, as compared to subjects without IBS.

In some embodiments, the subject is suffering from IBS and has increased levels of Aeromonas, as compared to subjects without IBS.

In some embodiments, the enteritis is caused by radiation therapy or chemotherapy.

In some embodiments, a gastrointestinal (GI) cleanser is administered to a subject prior to administration of the composition.

In some embodiments, the gastrointestinal cleanser is administered between about 1 to about 90 days prior to administration of the composition.

In some embodiments, administration of the gastrointestinal cleanser is between about 1 to about 60 days; between about 1 to about 30 days; between about 1 to about 24 days; between about 1 to about 14 days; between about 1 to about 10 days; between about 1 to about 7 days; between about 1 to about 5 days; between about 1 to about 4 days; between about 1 to about 3 days; or between about 1 to about 2 days prior to administration of the composition.

### DESCRIPTION OF THE DRAWINGS

FIG. 1 is a plot of the change in stool coliform counts during and after treatment with rifaximin.

FIG. 2 is an expanded view of the same plot indicating change in stool coliform counts during and after treatment with rifaximin.

FIG. 3 is a bar chart indicating total stool coliform counts by gastrointestinal segment and by various delivery vehicles for rifaximin administration.

FIG. 4 is a bar chart indicating total bacteria count as measured by quantitative PCR (qPCR) in the small intestine during and after completion of rifaximin administration.

FIG. 5 is a bar chart indicating total bacteria count as measured by quantitative PCR (qPCR) in the colon during and after completion of rifaximin administration.

FIG. 6 is a bar chart indicating total bacteria count as measured by quantitative PCR (qPCR) in the small intestine in controls relative to subjects treated by rifaximin (total).

FIG. 7 is a bar chart indicating total bacteria count as measured by quantitative PCR (qPCR) in the colon in controls relative to subjects treated by rifaximin (total).

FIG. 8 is a graph indicating the Staphylococcus spp. colony counts in rats at baseline and after 10 days of treatment with rifaximi.

FIG. 9 is a graph indicating the Staphylococcus spp. colony counts in rats by day during treatment with 200 mg rifaximin.

FIG. 10 is a bar chart comparing the presence of Staphylococcus spp. in the stool of rats after treatment with rifaximin or a placebo.

### DETAILED DESCRIPTION

Rifaximin (USAN, INN; see The Merck Index, XIII Ed., 8304, CAS No. 80621-81-4), (2S,16Z,18E,20S,21S,22R,23R,24R,25S,26S,27S,28E)-5,6,21,23,25 Pentahydroxy-27-methoxy-2,4,11,16,20,22,24,26-octamethyl-2,7-(epoxypentadeca-(1,11,13)trienimino)benzofuro(4,5-e)pyrido(1,2,-a)benzimidazole-1,15(2H)-dione,25-acetate), is a semi-synthetic antibiotic produced from rifamycin O. Rifaximin is a molecule belonging to the rifamycin class of antibiotics, e.g., a pyrido-imidazo rifamycin. Rifaximin exerts a broad antibacterial activity, for example, in the gastrointestinal tract against localized gastrointestinal bacteria that cause infectious diarrhea, irritable bowel syndrome, small intestinal bacterial overgrowth, Crohn's disease, and/or pancreatic insufficiency.

Rifaximin is also described in Italian Patent IT 1154655 and EP 0161534. EP patent 0161534 discloses a process for rifaximin production using rifamycin O as the starting material (The Merck Index, XIII Ed., 8301). U.S. Pat. No. 7,045,620 B1 discloses polymorphic forms of rifaximin, as do U.S. Ser. No. 11/658,702; U.S. Ser. No. 61/031,329; U.S. Ser. No. 12/119,622; U.S. Ser. No. 12/119,630; U.S. Ser. No. 12/119,612; U.S. Ser. No. 12/119,600; U.S. Ser. No. 11/873,841; Publication WO 2006/094662; and U.S. Ser. No. 12/393,012. The applications and patents referred to here are incorporated herein by reference in their entirety for all purposes.

A rifamycin class antibiotic is, for example, a compound having the structure of Formula I:

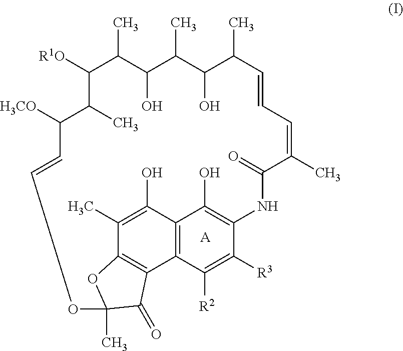

wherein

ring A may exist in tautomeric forms A¹ or A²

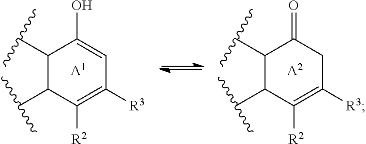

R¹ is hydrogen or acetyl; and

R² and R³ are each independently hydrogen, (C₁₋₄)alkyl, benzyloxy, mono- and di-(C₁₋₃)alkylamino-(C₁₋₄) alkyl, (C₁₋₃)alkoxy-(C₁₋₄)alkyl, hydroxymethyl, hydroxy-(C₂₋₄)-alkyl, or nitro; or

R² and R³ taken together to which the atoms they are attached form an optionally substituted heteroaryl or optionally substituted heterocyclyl.

Also described herein is a rifamycin compound of Formula (I) selected from 4-deoxy-4′-methyl-pyrido[1′,2′-1,2]imidazo[5,4-c]rifamycin SV and 4-deoxy-pyrido[1′,2′:1,2]imidazo[5,4-c]rifamycin SV.

Rifaximin is a compound having the structure of Formula II:

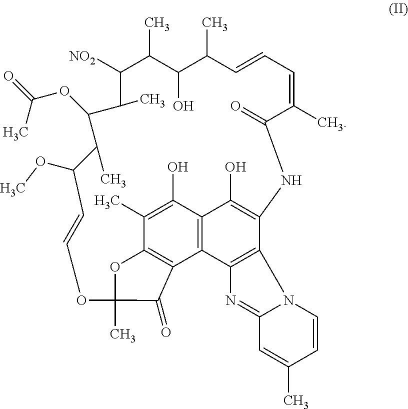 [SMILES: COC1/C=C/OC2(C)Oc3c(C)c(O)c4c(O)c(c5c(nc6cc(C)ccn65)c4c3C2=O)NC(=O)/C(C)=C\C=C\C(C)C(O)C(C)C([N+](=O)[O-])C(C)C(OC(C)=O)C1C]

Without wishing to be bound by any particular scientific theories, rifaximin acts by binding to the beta-subunit of the bacterial deoxyribonucleic acid-dependent ribonucleic acid (RNA) polymerase, resulting in inhibition of bacterial RNA synthesis. It is active against numerous gram (+) and (−) bacteria, both aerobic and anaerobic. In vitro data indicate rifaximin is active against species of Staphylococcus, Streptococcus, Enterococcus, and Enterobacteriaceae.

“Rifaximin”, as used herein, includes solvates and polymorphous forms of the molecule, including, for example, Form α, Form β, Form γ, Form δ, Form ε, Form ζ, Form η, Form ι, Form kappa, Form theta, Form mu, Form omicron, Form pi, mesylate Form or amorphous Forms of rifaximin. These forms are described in more detail, for example, in EP 05 004 695.2, filed 3 Mar. 2005; U.S. Pat. No. 7,045,620; U.S. Pat. No. 7,612,199; U.S. Pat. No. 7,709,634; U.S. Pat. No. 7,915,275; U.S. Pat. No. 8,067,429; U.S. Pat. No. 8,193,196; U.S. Pat. No. 8,227,482; G. C. Viscomi, et al., CrystEngComm, 2008, 10, 1074-1081 (April 2008), US Patent Publication 2010/0174064, US Patent Publication 2009/0028940, US Patent Publication 2005/0272754, U.S. Patent Publication No. 2012/0108620. Each of these references is hereby incorporated by reference in entirety.

“Polymorphs” or “polymorphic forms” as used herein, refer to the occurrence of different crystalline forms of a single compound in distinct hydrate status, e.g., a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as solubility profiles, melting point temperatures, hygroscopicity, particle shape, density, flowability, compatibility and/or x-ray diffraction peaks. The solubility of each polymorph may vary, thus, identifying the existence of pharmaceutical polymorphs is essential for providing pharmaceuticals with predictable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. For a general review of polymorphs and the pharmaceutical applications of polymorphs see G. M. Wall, Pharm Manuf. 3, 33 (1986); J. K. Haleblian and W. McCrone, J Pharm. Sci., 58, 911 (1969); and J. K. Haleblian, J. Pharm. Sci., 64, 1269 (1975), all of which are incorporated herein by reference. As used herein, the term polymorph is occasionally used as a general term in reference to the forms of rifaximin and include within the context, salt, hydrate, polymorph and amorphous forms of rifaximin disclosed herein. This use depends on context and will be clear to one of skill in the art. Exemplary polymorphic forms of rifaximin useful in the methods and kits as disclosed herein are set forth in the published patent applications set forth above.

Medicinal preparations may contain gastrointestinal specific antibiotics together with usual excipients, discussed infra.

“GI specific antibiotic,” and “GI antibiotic” as used herein include antibiotic known to have an effect on GI disease. For example, a rifamycin class antibiotic (e.g., rifaximin), neomycin, metronidazole, teicoplanin, ciprofloxacin, doxycycline, tetracycline, augmentin, cephalexin, penicillin, ampicillin, kanamycin, rifamycin, vancomycin, and combinations thereof are useful GI specific antibiotics. Even more preferable are GI specific antibiotics with low systemic absorption, for example, rifaximin Low systemic absorption includes, for example, less than 10% absorption, less than 5% absorption, less than 1% absorption and less than 0.5% absorption. Low systemic absorption also includes, for example, from between about 0.01-1% absorption, from between about 0.05-1% absorption, from between about 0.1-1% absorption, from between about 1-10% absorption, or from between about 5-20% absorption.

“Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with rifaximin, where the untreated subjects have, or are subject to developing, the same or similar disease, condition, symptom or the like. Amelioration of a disease, condition, symptom or assay parameter may be determined subjectively or objectively, e.g., self assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of CDI in a subject. Amelioration may be transient, prolonged or permanent or it may be variable at relevant times during or after a GI specific antibiotic is administered to a subject or is used in an assay or other method described herein or a cited reference, e.g., within timeframes described infra, or about 1 hour after the administration or use of a GI specific antibiotic to about 7 days, 2 weeks, 28 days, or 1, 3, 6, 9 months or more after a subject(s) has received such treatment.

The “modulation” of, e.g., a symptom, level or biological activity of a molecule, or the like, refers, for example, that the symptom or activity, or the like is detectably increased or decreased. Such increase or decrease may be observed in treated subjects as compared to subjects not treated with a GI specific antibiotic, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively, e.g., by the subject's self assessment, by a clinician's assessment or by conducting an appropriate assay or measurement, including, e.g., quality of life assessments or suitable assays for the level or activity of molecules within a subject. Modulation may be transient, prolonged or permanent or it may be variable at relevant times during or after a GI specific antibiotic is administered to a subject or is used in an assay or other method described herein or a cited reference, e.g., within times descried infra, or about 1 hour of the administration or use of a GI specific antibiotic to about 2 weeks, 28 days, 3, 6, 9 months or more after a subject(s) has received a GI specific antibiotic.

The term “modulate” may also refer to increases or decreases in the activity of a cell in response to exposure to a GI specific antibiotic, e.g., the inhibition of proliferation and/or induction of differentiation of at least a sub-population of cells in an animal such that a desired end result is achieved, e.g., a therapeutic result of GI specific antibiotic used for treatment may increase or decrease over the course of a particular treatment.

The language “a prophylactically effective amount” of a compound refers to an amount of a compound of formula I, formula II, or otherwise described herein which is effective, upon single or multiple dose administration to the subject, in preventing or treating BD.

As used herein, “subject” includes organisms which are capable of suffering from, or susceptible to, a disease or symptom that is treatable by a rifamycin class antibiotic (e.g., rifaximin) or who could otherwise benefit from the administration of a rifamycin class antibiotic (e.g., rifaximin) as described herein, such as human and non-human animals. Preferred human animals include human subjects. The term “non-human animals” includes all vertebrates, e.g., mammals, e.g., rodents, e.g., mice, and non-mammals, such as non-human primates, e.g., sheep, dog, cow, chickens, amphibians, reptiles, etc. Susceptible to a bowel disorder is meant to include a subject at risk of developing a bowel disorder, a subject who is in remission from the treated disease or symptom, a subject who may relapse from the treated disease or system, a subject that has been exposed to a bacterial infection, e.g., physicians or nurses.

The language “a prophylactically effective amount” of a compound refers to an amount of a compound of formula I, formula II, or otherwise described herein which is effective, upon single or multiple dose administration to the subject, in preventing or treating CDI.

The term “administration” or “administering” includes routes of introducing a GI specific antibiotic to a subject to perform their intended function. Examples of routes of administration that may be used include injection, oral, inhalation, vaginal, rectal and transdermal. The pharmaceutical preparations may be given by forms suitable for each administration route. For example, these preparations are administered in tablets or capsule form, by injection, inhalation, eye lotion, eye drops, ointment, suppository, etc. administration by injection, infusion or inhalation; topical by lotion or ointment; and rectal by suppositories. Oral administration is preferred. The injection can be bolus or can be continuous infusion. Depending on the route of administration, a GI specific antibiotic can be coated with or disposed in a selected material to protect it from natural conditions that may detrimentally affect its ability to perform its intended function. A GI specific antibiotic can be administered alone, or in conjunction with either another agent or agents as described above or with a pharmaceutically-acceptable carrier, or both. A GI specific antibiotic can be administered prior to the administration of the other agent, simultaneously with the agent, or after the administration of the agent. Furthermore, a GI specific antibiotic can also be administered in a proform, which is converted into its active metabolite, or more active metabolite in vivo.

Administration “in combination with” one or more further therapeutic agents includes simultaneous (concurrent) and consecutive administration in any order.

As will be readily apparent to one skilled in the art, the useful in vivo dosage to be administered and the particular mode of administration will vary depending upon the age, weight and mammalian species treated, the particular compounds employed, and/or the specific use for which these compounds are employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved.

The term “obtaining” as in “obtaining a GI specific antibiotic” is intended to include purchasing, synthesizing or otherwise acquiring a GI specific antibiotic.

The language “a prophylactically effective amount” of a compound refers to an amount of a GI specific antibiotic which is effective, upon single or multiple dose administration to the subject, in preventing or treating CDI.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

As used herein, “durability of response” includes for example, adequate relief of symptoms after removal of treatment, continuous adequate relief of symptoms after removal of treatment, or response that is greater than or superior to placebo response. A response by a subject may be considered durable, for example, if they have a response to the rifamycin class antibiotic after removal from treatment. The duration of response, may be, for example, 2 days, 7 days, two weeks, 3 weeks, 4 weeks, 12 weeks, between about 1 week and about 24 weeks or longer. The response may be measured, for example using one or more of the methods outlined below, including, for example, a subject's subjective assessment of their symptoms or a healthcare provider's or caretaker's assessment of a subject's symptoms.

Embodiments are directed to a method of reducing, reducing the amount of, or reducing the development of rifampicin-resistant Staphylococcus spp. organisms in a subject in need thereof, wherein the method includes administering a composition comprising a therapeutically effective amount of rifaximin to the subject. In some embodiments, administration of the composition results in a reduction in the number of rifampicin-resistant Stapholococcus spp. compared to a pre-treatment baseline number. In some embodiments, the reduction in the number of rifampicin-resistant Staphylococcus spp. relative to baseline is determined by using any method known to one of skill in the art to measure such a value. For example, the reduction in the number of rifampicin-resistant Staphylococcus spp. relative to baseline can be determined by culturing stool samples obtained from the subject prior and subsequent to administration of the composition.

Embodiments also relate to a method of reducing, reducing the amount of, or reducing the development of, rifampicin-resistant Staphylococcus spp. organisms in a subject, wherein the method comprises administering rifaximin to the subject in need of antibiotic treatment for a condition. In some embodiments, the condition is one selected from the group of: an inflammatory bowel disease (IBD), travelers' diarrhea (TD), hepatic encephalopathy (HE), minimal hepatic encephalopathy, irritable bowel syndrome (IBS), diarrhea-predominant irritable bowel syndrome (d-IBS), non-constipation-predominant irritable bowel syndrome (non-c-IBS), a Clostridium difficle infection (CDI), fibromyalgia (FM), chronic fatigue syndrome (CFS), depression, attention deficit/hyperactivity disorder (ADHD), multiple sclerosis (MS), systemic lupus erythematosus (SLE), restless leg syndrome, dermal infections, small intestinal bacterial overgrowth, chronic pancreatitis, pancreatic insufficiency, diverticulitis (or diverticular disease), enteritis, colitis, skin infections, mucous membrane disorders, pouchitis, vaginal infections, anal fissures, ear infections, lung infections, periodontal conditions, rosacea, and other infections of the skin and/or other related conditions. In some embodiments, the inflammatory bowel disease is Crohn's disease or ulcerative colitis. In some embodiments, the enteritis is caused by radiation therapy or chemotherapy.

In some embodiments, a gastrointestinal (GI) cleanser is administered to a subject prior to administration of the composition.

In some embodiments, the gastrointestinal cleanser is administered between about 1 to about 90 days prior to administration of the composition. In some embodiments, the administration of the gastrointestinal cleanser is between about 1 to about 60 days; between about 1 to about 30 days; between about 1 to about 24 days; between about 1 to about 14 days; between about 1 to about 10 days; between about 1 to about 7 days; between about 1 to about 5 days; between about 1 to about 4 days; between about 1 to about 3 days; or between about 1 to about 2 days prior to administration of the composition.

In some embodiments, the gastrointestinal cleanser comprises one or more of a PEG-based composition or a sodium phosphate-based composition. In some embodiments, the gastrointestinal cleanser comprises polyethylene glycol (PEG), sodium sulfate, sodium chloride, potassium chloride, and ascorbic acid. In some embodiments, the gastrointestinal cleanser comprises sodium phosphate monobasic, sodium phosphate dibasic, microcrystalline cellulose, colodial silicon dioxide, and magnesium stearate.

Rifaximin may be used in various treatment regimes. These regimes may vary depending upon the subject and the type of treatment.

Rifaximin may be administered, for example, twice a day, three times a day, or four times or more often as necessary per day. Rifaximin may be administered in doses, for example of from about between 25 mg once daily to about 3000 mg TID. In some embodiments, the subject is administered rifaximin at a dose of about 50 mg to about 6000 mg per day. For example, rifaximin can be administered in daily doses of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, In some embodiments, rifaximin can be administered in daily doses of about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg, In some embodiments, rifaximin can be administered in daily doses of about 550 mg, about 600 mg, about 650 mg, about 700 mg, about 750 mg, about 800 mg, about 850 mg, about 900 mg, about 950 mg, or about 1000 mg. In some embodiments, rifaximin can be administered in daily doses of about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, or about 3000 mg, In some embodiments, rifaximin can be administered in doses of about 25 mg BID, about 30 mg BID, about 35 mg BID, about 40 mg BID, about 45 mg BID, about 50 mg BID, about 55 mg BID, about 60 mg BID, about 65 mg BID, about 70 mg BID, about 75 mg BID, about 80 mg BID, about 85 mg BID, about 90 mg BID, about 95 mg BID, or about 100 mg BID, In some embodiments, rifaximin can be administered in doses of about 125 mg BID, about 150 mg BID, about 175 mg BID, about 200 mg BID, about 225 mg BID, about 250 mg BID, about 275 mg BID, about 300 mg BID, about 325 mg BID, about 350 mg BID, about 375 mg BID, about 400 mg BID, about 425 mg BID, about 450 mg BID, about 475 mg BID, or about 500 mg BID, In some embodiments, rifaximin can be administered in doses of about 550 mg BID, about 600 mg BID, about 650 mg BID, about 700 mg BID, about 750 mg BID, about 800 mg BID, about 850 mg BID, about 900 mg BID, about 950 mg BID, or about 1000 mg BID. In some embodiments, rifaximin can be administered in doses of about 1100 mg BID, about 1200 mg BID, about 1300 mg BID, about 1400 mg BID, about 1500 mg BID, about 1600 mg BID, about 1700 mg BID, about 1800 mg BID, about 1900 mg BID, about 2000 mg BID, about 2100 mg BID, about 2200 mg BID, about 2300 mg BID, about 2400 mg BID, about 2500 mg BID, about 2600 mg BID, about 2700 mg BID, about 2800 mg BID, about 2900 mg BID or about 3000 mg BID, In some embodiments, rifaximin can be administered in doses of about 25 mg TID, about 30 mg TID, about 35 mg TID, about 40 mg TID, about 45 mg TID, about 50 mg TID, about 55 mg TID, about 60 mg TID, about 65 mg TID, about 70 mg TID, about 75 mg TID, about 80 mg TID, about 85 mg TID, about 90 mg TID, about 95 mg TID, or about 100 mg TID, In some embodiments, rifaximin can be administered in doses of about 125 mg TID, about 150 mg TID, about 175 mg TID, about 200 mg TID, about 225 mg TID, about 250 mg TID, about 275 mg TID, about 300 mg TID, about 325 mg TID, about 350 mg TID, about 375 mg TID, about 400 mg TID, about 425 mg TID, about 450 mg TID, about 475 mg TID, or about 500 mg TID, In some embodiments, rifaximin can be administered in doses of about 550 mg TID, about 600 mg TID, about 650 mg TID, about 700 mg TID, about 750 mg TID, about 800 mg TID, about 850 mg TID, about 900 mg TID, about 950 mg TID, or about 1000 mg TID. In some embodiments, rifaximin can be administered in doses of about 1100 mg TID, about 1200 mg TID, about 1300 mg TID, about 1400 mg TID, about 1500 mg TID, about 1600 mg TID, about 1700 mg TID, about 1800 mg TID, about 1900 mg TID, about 2000 mg TID, about 2100 mg TID, about 2200 mg TID, about 2300 mg TID, about 2400 mg TID, about 2500 mg TID, about 2600 mg TID, about 2700 mg TID, about 2800 mg TID, about 2900 mg TID or about 3000 mg TID, The rifaximin may be administered, for example, in tablet form, powdered form, liquid form or in capsules. In some embodiments, rifaximin can be administered in a time-released formulation.

In some embodiments, rifaximin is administered as a soluble solid dispersion. For example, rifaximin can be administered at between about 25-550 mg of soluble solid dispersion of rifaxmin Soluble solid dispersions of rifaximin are described in “FORMULATIONS OF RIFAXIMIN AND USES THEREOF,” U.S. Patent Publication No. 2012/0077835, which is incorporated herein by reference in its entirety.

In some embodiments, the rifaximin is administered to a subject from between about 1 week to about 6 weeks in duration, from between about 8 weeks to about 12 weeks in duration, or from between about 1 day to about 21 days in duration. In one embodiment, rifaximin is administered for 10 days. The rifaximin may be administered from between about 1 day and about 1 year, or from 1 week to about 52 weeks. In some embodiments, the rifaximin is administered from between about one week and about 24 months. The rifaximin may be administered intermittently or continuously during the course of treatment. Length of treatment may vary depending on the type and length of disease and the proper length of treatment may be easily determined by one of skill in the art having the benefit of this disclosure.

For any of the embodiments, rifaximin may be administered, for example, once daily, twice daily, three times daily, or four times daily (or more often as necessary for a particular subject) to a subject. In some embodiments, the methods comprise administering the rifaximin once daily to the subject because it may, for example, minimize the side effects and increase patient compliance. In some embodiments, rifaximin is administered twice and/or three times daily.

Dosages, according to certain preferred embodiments, range from between about 50 to about 6000 mg of rifaximin administered daily. For example, a dose of 400 mg may be administered to a subject three times daily, or a dose of 550 mg may be administered to a subject twice daily. Other appropriate dosages for the methods as disclosed herein may be determined by health care professionals or by the subject. The amount of rifaximin administered daily may be increased or decreased based on the weight, age, health, sex or medical condition of the subject. One of skill in the art would be able to determine the proper dose for a subject based on this disclosure.

According to certain embodiments, rifaximin may be administered in combination with other compounds, including for example, chemotherapeutic agents, anti-inflammatory agents, anti-pyretic agents radiosensitizing agents, radioprotective agents, urologic agents, anti-emetic agents, and/or anti-diarrheal agents. For example, cisplatin, carboplatin, docetaxel, paclitaxel, flurouracil, capecitabine, gemcitabine, irinotecan, topotecan, etoposide, mitomycin, gefitinib, vincristine, vinblastine, doxorubicin, cyclophosphamide, celecoxib, rofecoxib, valdecoxib, ibuprofen, naproxen, ketoprofen, dexamethasone, prednisone, prednisolone, hydrocortisone, acetaminophen, misonidazole, amifostine, tamsulosin, phenazopyridine, ondansetron, granisetron, alosetron, palonosetron, promethazine, prochlorperazine, trimethobenzamide, aprepitant, diphenoxylate with atropine, and/or loperamide.

Embodiments of the invention also include pharmaceutical compositions comprising an effective amount of a rifamycin class antibiotic (e.g., rifaximin or a rifaximin polymorph) described herein and a pharmaceutically acceptable carrier.

In some embodiments, the pharmaceutical composition comprises rifaximin or any polymorphic form thereof and a pharmaceutically acceptable carrier. That is, formulations may contain only one polymorph or may contain a mixture of more than one polymorph. Polymorph, in this context, refers to any physical form, hydrate, acid, salt or the like of rifaximin Mixtures may be selected, for example on the basis of desired amounts of systemic adsorption, dissolution profile, desired location in the digestive tract to be treated, and the like. The pharmaceutical composition further comprises excipients, for example, one or more of a diluting agent, binding agent, lubricating agent, disintegrating agent, coloring agent, flavoring agent or sweetening agent. Compositions may be formulated for selected coated and uncoated tablets, hard and soft gelatin capsules, sugar-coated pills, lozenges, wafer sheets, pellets and powders in sealed packet. For example, compositions may be formulated for topical use, for example, ointments, pomades, creams, gels and lotions.

In some embodiments, the rifamycin class antibiotic (e.g., rifaximin) is administered to the subject using a pharmaceutically-acceptable formulation, e.g., a pharmaceutically-acceptable formulation that provides sustained delivery of the rifamycin class antibiotic (e.g., rifaximin) to a subject for at least 12 hours, 24 hours, 36 hours, 48 hours, one week, two weeks, three weeks, or four weeks after the pharmaceutically-acceptable formulation is administered to the subject.

In some embodiments, these pharmaceutical compositions are suitable for topical or oral administration to a subject. In some embodiments, as described in detail below, the pharmaceutical compositions may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those rifamycin class antibiotic (e.g., rifaximin) described herein, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a rifamycin class antibiotic (e.g., rifaximin) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1% to about 99% of active ingredient, preferably from about 5% to about 70%, most preferably from about 10% to about 30%.

Liquid dosage forms for oral or rectal administration of the rifamycin class antibiotic (e.g., rifaximin) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active rifamycin class antibiotic (e.g., rifaximin) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions for rectal or vaginal administration may be presented as a suppository, which may be prepared by mixing one or more rifamycin class antibiotic (e.g., rifaximin) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent. Compositions which are suitable for vaginal administration can include pessaries, tampons, creams, gels, pastes, foams or spray formulations containing such carriers as are known in the art to be appropriate.

Dosage forms for the topical or transdermal administration of a rifamycin class antibiotic (e.g., rifaximin) can include powders, sprays, ointments, pastes, creams, lotions, gels, solutions, patches and inhalants. The active rifamycin class antibiotic (e.g., rifaximin) may be mixed under sterile conditions with a pharmaceutically-acceptable carrier, and with any preservatives, buffers, or propellants which may be beneficial.

The ointments, pastes, creams and gels may contain, in addition to rifamycin class antibiotic (e.g., rifaximin), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a rifamycin class antibiotic (e.g., rifaximin), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The rifamycin class antibiotic (e.g., rifaximin) can be alternatively administered by aerosol. This is accomplished, for example, by preparing an aqueous aerosol, liposomal preparation or solid particles containing the compound. A non-aqueous (e.g., fluorocarbon propellant) suspension could be used. Sonic nebulizers are preferred because they minimize exposing the agent to shear, which can result in degradation of the compound.

Examples of suitable aqueous and non-aqueous carriers which may be employed in the pharmaceutical compositions can include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, to prolong the effect of a drug, it is desirable to alter the absorption of the drug. This may be accomplished by the use of a liquid suspension of crystalline, salt or amorphous material having poor water solubility. The rate of absorption of the drug may then depend on its rate of dissolution which, in turn, may depend on crystal size and crystalline form. Alternatively, delayed absorption of a drug form is accomplished by dissolving or suspending the drug in an oil vehicle.

When the rifamycin class antibiotic (e.g., rifaximin) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the rifamycin class antibiotic (e.g., rifaximin), which may be used in a suitable hydrated form and/or pharmaceutical compositions as disclosed herein, are formulated into pharmaceutically-acceptable dosage forms by methods known to those of skill in the art.

Actual dosage levels and time course of administration of the active ingredients in the pharmaceutical compositions as disclosed herein may be varied so as to obtain an amount of the active ingredient which is effective to achieve the desired therapeutic response for a particular patient, composition, and mode of administration, without being toxic to the patient. An exemplary dose range is from 25 to 3000 mg per day.

In combination therapy treatment, both the compounds as disclosed herein and the other drug agent(s) are administered to mammals (e.g., humans, male or female) by methods. The agents may be administered in a single dosage form or in separate dosage forms. Effective amounts of the other therapeutic agents are well known to those skilled in the art. However, it is well within the skilled artisan's purview to determine the other therapeutic agent's effective-amount range. In embodiments in which another therapeutic agent is administered to an animal, the effective amount of the compound is less than its effective amount in case the other therapeutic agent is not administered. In some embodiments, the effective amount of the agent is less than its effective amount in case the compound is not administered. In this way, undesired side effects associated with high doses of either agent may be minimized Other potential advantages (including without limitation improved dosing regimens and/or reduced drug cost) will be apparent to those skilled in the art.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same patient's visit.

In certain embodiments, one or more of the rifamycin class antibiotic (e.g., rifaximin) and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, e.g., the cycle in order to reduce the development of resistance to one of the therapies, to avoid or reduce the side effects of one of the therapies, and/or to improve the efficacy of the therapies.

In certain embodiments, the administration of the same compounds may be repeated and the administrations may be separated by at least about 1 day, 2 days, 3 days, 5 days, 10 days, 15 days, 30 days, 45 days, 3 weeks, 4 weeks, 5 weeks, 6 weeks, 12 weeks, 2 months, 75 days, 3 months, or at least 6 months. In other embodiments, the administration of the same therapy (e.g., prophylactic or therapeutic agent) other than a rifamycin class antibiotic (e.g., rifaximin) may be repeated and the administration may be separated by at least at least 1 day, 2 days, 3 days, 5 days, 10 days, 15 days, 30 days, 45 days, 2 months, 75 days, 3 months, or at least 6 months. In one embodiment, a label on a rifamycin class antibiotic may instruct, for example, do not repeat more often than every 6 weeks. In another embodiment, a label on a rifamycin class antibiotic may instruct, for example, do not repeat more often than every 3 weeks. In another embodiment, a label on a rifamycin class antibiotic may instruct, for example, do not repeat more often than every 3-12 weeks. Included within ranges given herein for dosage or administration are any value within the range.

In some embodiments, retreatment is efficacious in combination with the methods disclosed herein. For example, methods as described herein may further comprise determining symptom relief in a subject and administering a second course of rifaximin treatment if symptoms remain unresolved.

Kits are also provided herein, for example, kits for treating a disease, symptom, or infection with rifaximin in a subject. The kits may contain, for example, a polymorph or amorphous form of rifaximin and instructions for use. The instructions for use may contain prescribing information, dosage information, storage information, and the like.

In some embodiments, the label describes adverse events comprising one or more of infections and infestations, gastrointestinal disorders, nervous system disorders, and musculoskeletal and connective tissue disorders.

In some embodiments, the label describes a length of treatment with the rifamycin class antibiotic, whereby a subject is selected as responding to treatment if a healthcare professional prescribes the rifamycin class antibiotic according to the label instructions.

In some embodiments, the label describes a length of treatment with the rifamycin class antibiotic, whereby a subject is removed from treatment if a healthcare professional prescribes the rifamycin class antibiotic according to the label instructions.

Packaged compositions are also provided, and may comprise a therapeutically effective amount of one or more of a one or more of an amorphous form, Form α, Form β, Form γ, Form δ, Form ε, Form ζ, Form mu, Form omicron, Form kappa, Form iota, or Form η polymorph of rifaximin of rifaximin and a pharmaceutically acceptable carrier or diluent, wherein the composition is formulated for treating a subject suffering from or susceptible to a bowel disorder, and packaged with instructions to treat a subject suffering from or susceptible to a bowel disorder.

### EXAMPLES

It should be appreciated that embodiments of the invention as disclosed herein should not be construed to be limited to the example, which is now described; rather, the embodiments can be construed to include any and all applications provided herein and all equivalent variations within the skill of the ordinary artisan.

### Example 1

### Experimental Methods

### Baseline Measurements

Adult male Sprague-Dawley rats were acquired and quarantined for 5 days. During this time, chow was standardized to ensure all animals had identical feeding type.

After this equilibration period, fresh stool was collected from each rat (n=30) by anal stimulation. The stool collected on Day 0 was homogenized and plated by serial dilution with 1XPBS (Phosphate Buffered Saline) on Blood Agar with Phenylethyl Alcohol (PEA) to select for the presence of the Staphylococcal spp. Similarly, homogenized stool was serially plated on MacConkey agar to select for and determine coliform counts in the stool. All plates were incubated for 24 hours at 37° C. Based on serial stool dilution and colony counts, the baseline stool levels of Staphylococcus spp. and coliforms were determined

### Rifaximin Gavage and Stool Coliform Pattern

Rats were divided into 3 treatment groups and gavaged daily for 10 days with or without high dose rifaximin Group 1 was gavaged with PBS alone, Group 2 was gavaged daily with rifaximin 200 mg in PBS. Groups 1 and 2 were euthanized after the 10 days of daily gavage. Group 3 was gavaged daily with rifaximin 200 mg in PBS and housed for 30 days following completion of rifaximin before euthanasia. During the rifaximin treatment, fresh stool was collected daily as described above. Again, stool was homogenized and plated by serial dilution on MacConkey and PEA agar to determine coliform and Staphylococcus spp. counts during the treatment. After the completion of 10 days of treatment, 10 rats were euthanized for bacterial quantitation by culture and qPCR throughout the intestinal tract. The remaining 10 rats were followed for 30 days after completing rifaximin. These rats had interval stool culture for quantitation of Staphylococcus spp. and coliforms and the determination of recovery time of stool flora if any.

### Luminal Quantitation of Bacteria

For the examination of luminal bacterial counts, rats were euthanized and dissected after 10 days of PBS (n=10) (control), after 10 days of rifaximin (n=10) and 30 days post rifaximin (n=10). During the dissection, pre-specified segments of duodenum, jejunum, ileum, cecum and left colon were ligated and resected as previously described (Pimentel et al. 2008. Dig Dis Sci 53:982-989). Luminal contents were extracted from each segment. For culture of coliforms, serial dilutions were again prepared and plated on MacConkey agar then incubated and counted. In addition, luminal contents were also used to determine total bacterial counts by qPCR.

To quantify bacteria in the luminal contents, qPCR was used. DNA was isolated from luminal contents of the duodenal, jejunal, ileal, cecal and left colon samples using commercially available kits. Bacterial universal primers (Mohammadi et al. 2003. J Clin Microbiol 41(10):4796-4798) were used to amplify the 16S rRNA gene from DNA using a commercially available RT-PCR detection system and optical grade 96-well plates. Samples were run in duplicate. To generate standard curves, the Ct values were analyzed from ten-fold dilutions of lysed Escherichia coli cultures. E. coli from a number of laboratory strains were pooled and grown in LB media.

### Rifampicin Susceptibility Testing

Rifampicin susceptibility was tested in Staphylococcus spp. isolated from stool of rats before and after rifaximin treatment for 10 days. From PEA plates, 30 random recognizable Staphylococcus colonies were picked for baseline cultures, suspended in PBS and spread on PEA agar plates to create a lawn. A commercially available rifampicin test strip was added to each lawn to detect rifampicin resistance. The range of rifampicin mean inhibitory concentrations (MICs) detected by E-test was 0.002 to 32 μg/mL. Based on manufacturer's instruction, growth at <2 ug/mL was considered sensitive, growth at 2-4 ug/mL was considered intermediate resistance to rifampicin, and growth at >4 ug/mL was considered resistant to rifampicin. After 10 days of rifaximin, stool was again plated on PEA and another 30 staphylococcal colonies were picked for producing a lawn to analyze rifampicin resistance using the applied test strip. Since no significant Staphylococcus spp. colonies remained on day 30 post rifaximin treatment, sensitivity testing could not be assessed.

### Data Analysis

To compare stool colony counts of bacteria or qPCR between groups, a Mann-Whitney U test was used, and data were expressed as median due to data being non-normal. When comparing colony counts before and after rifaximin, a Wilcoxon Rank sum test for matched pairs was used. Significance was noted as a P-value<0.05. In comparing trends in the counts from control to rifaximin day 10 and 30 days post rifaximin, data were log transformed to normalize the data and compared by Kruskal-Wallis test.

### IBS Model

Following acute infection with C. jejuni, rats develop a phenotype of IBS and small intestinal bacterial overgrowth. For the examination of antibiotic resistance following rifaximin treatment in subject with IBS, 100 Sprague-Dawley rats were gavaged with 108 cfu of C. jejuni 81-176 within 30 minutes of gavage with a bicarbonate solution using a ball-tipped gavage needle. After gavage rats were followed with daily stool culture for C. jejuni for seven consecutive days to verify infection. In addition, stool was characterized on the basis of consistency.

Thirty days from gavage, stool samples were collected on two consecutive days to confirm absence of C. jejuni in stool. Once rats were confirmed to have cleared C. jejuni, they were housed in common conditions for a period of three months.

Three months after clearance of C. jejuni, the rats had a five day morning stool collection. This stool collection was used to determine stool percentage wet weight in the absence of C. jejuni, and stool consistence on a 5 point Bristol like stool scale.

The rats were then randomized and received either a 200 mg dose of rifaximin in PBS or PBS alone daily for 10 days. During the ten day period, stool samples were collected daily to assess bacteria counts, stool consistency and stool wet weight. Upon completion of treatment with rifaximin, the rats were monitored for an additional seven days. During the final five days, stool samples were collected and examined for consistency, wet weight and bacterial counts.

Finally, rats were euthanized by CO₂ asphyxiation and dissected. The dissection included ligation of sections of the premeasured ileum, jejunum, duodenum, cecum, transverse colon, left colon and rectum in order to preserve the luminal contents for the evaluation of the internal microflora

The effect of rifaximin on Staphylococcal species was tested and antibiotic resistance was examined. The contents of the ligated bowel segments were examined to determine the quantity and type of bacteria by segment and evaluate expression of mucosal cytokines.

### Example 2

### Effect of Rifaximin on Stool Gram-Positive Bacteria Such as Staphylococcus

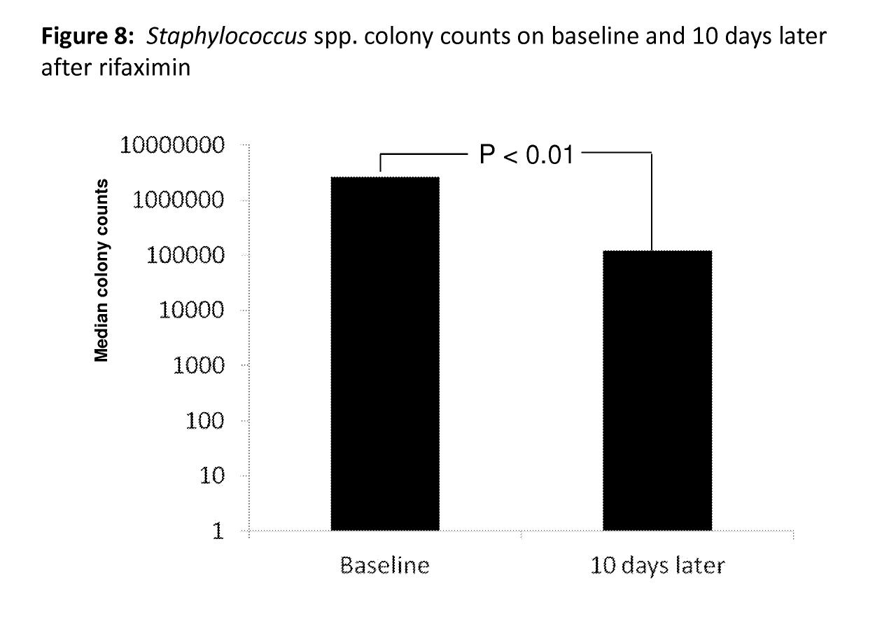

A total of 20 male Sprague-Dawley rats were assessed (Example 1). At baseline, rats had a median of 5.50×10⁵ cfu/ml (range=0-1.96×10⁶ cfu/ml) Staphylococcus spp (total of S. aureus and coag-Staphylococcus). 18/20 rats had detectable S. aureus in stool at baseline, but all rats had coagulase negative Staphylococcus. After 10 days of rifaximin, the median total count dropped significantly to 1.20×10⁵ cfu/ml (range=0-8.6×10⁵ cfu/ml) (p<0.01 by Wilcoxon Rank Sum test for matched pairs) (FIG. 8). On day 10 of rifaximin treatment, five (5) rats had detectable S. aureus in stool but at a low level of 2×10³ cfu/ml. On a time course, Staphylococcus spp were seen to diminish significantly in stool by day 3. Before rifaximin treatment and at day 10 of rifaximin treatment, 30 random colonies of Staphylococcal species were picked and plated for testing rifampicin resistance. At baseline (before rifaximin), two (2) colonies of Staphylococcus spp were resistant, and five (5) colonies exhibited intermediate resistance to rifampicin (Table 1). However, after conclusion of rifaximin treatment, no colonies were resistant, and only one (1) colony exhibited intermediate resistance to rifampicin. The mean inhibitory concentration (MIC) for rifampicin was 1.1±1.6 ug/mL for baseline and 0.91±0.52 ug/mL after 10 days of high dose rifaximin (P=0.63).

TABLE 1
Staphylococcal resistance to rifampin before
and after 10 days of gavage with rifaximin

| | Sensitive | Intermediate | Resistant |
|---|---|---|---|
| [n(%)] | (<2 ug/mL) | (2-4 ug/mL) | (>4 ug/mL) |
| Baseline [n(%)] | 22 (75.8%) | 5 (17.2%) | 2 (6.9%) |
| Day 10 rifaximin | 29 (96.6%) | 1 (3.3%) | 0 |

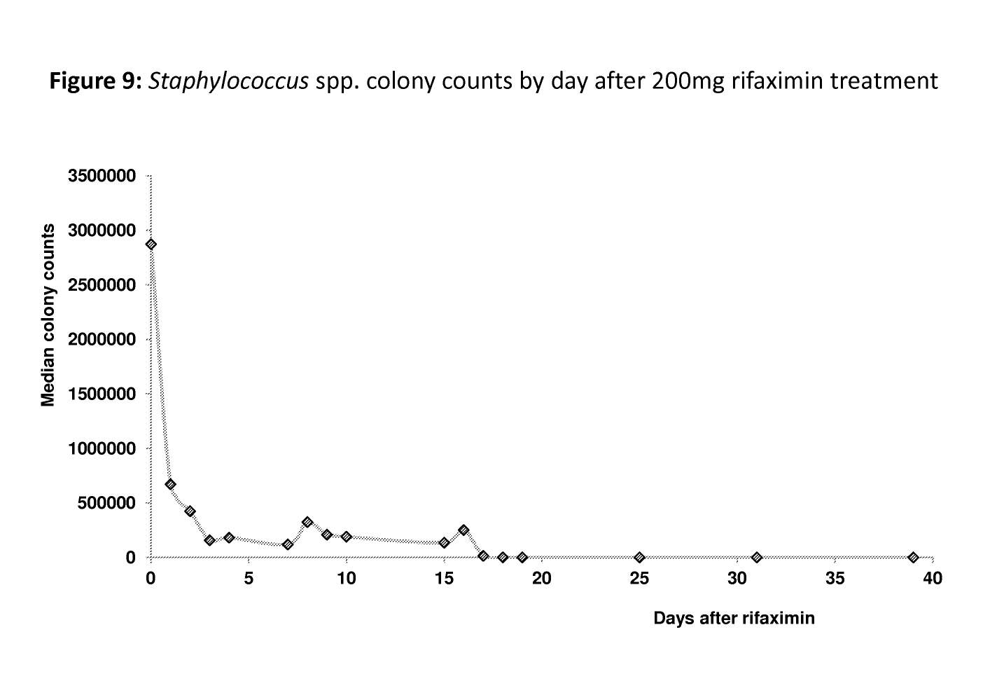

The results indicate that stool from Sprague-Dawley rats have a large quantity of Staphylococcus spp in their stool, predominantly coag-Staphylococcus. Treatment with rifaximin reduces Staphylococcus spp counts in the stool. Furthermore, although some Staphylococcus spp persist after high dose rifaximin, none of the species demonstrate resistance to rifampicin despite rare cases of resistance observed prior rifaximin treatment. Thus, rifaximin does not appear to select for rifampicin resistance in Staphylococcus spp after a 10 day course and may impair or reduce numbers of rifampicin-resistant Staphylococus spp. This is demonstrated by the fact that after treatment with rifaximin, there was less evidence of rifampicin resistance than pre-treatment. Rifaximin also appears to reduce Staphylococcal colony counts in total. In particular, after 30 days, there were insufficient Staphylococcus spp. colonies in any of the rats to analyze (FIG. 9).

Accordingly, provided herein are methods of administering rifaximin to a subject in need thereof, wherein administration of rifaximin reduces the number of resistant Staphylococcal spp in the subject. In some embodiments, administration of rifaximin has no effect on MIC of rifampicin Staphylococcal spp in the subject.

### Example 3

### Coliform Culture of Stool with and without Rifaximin

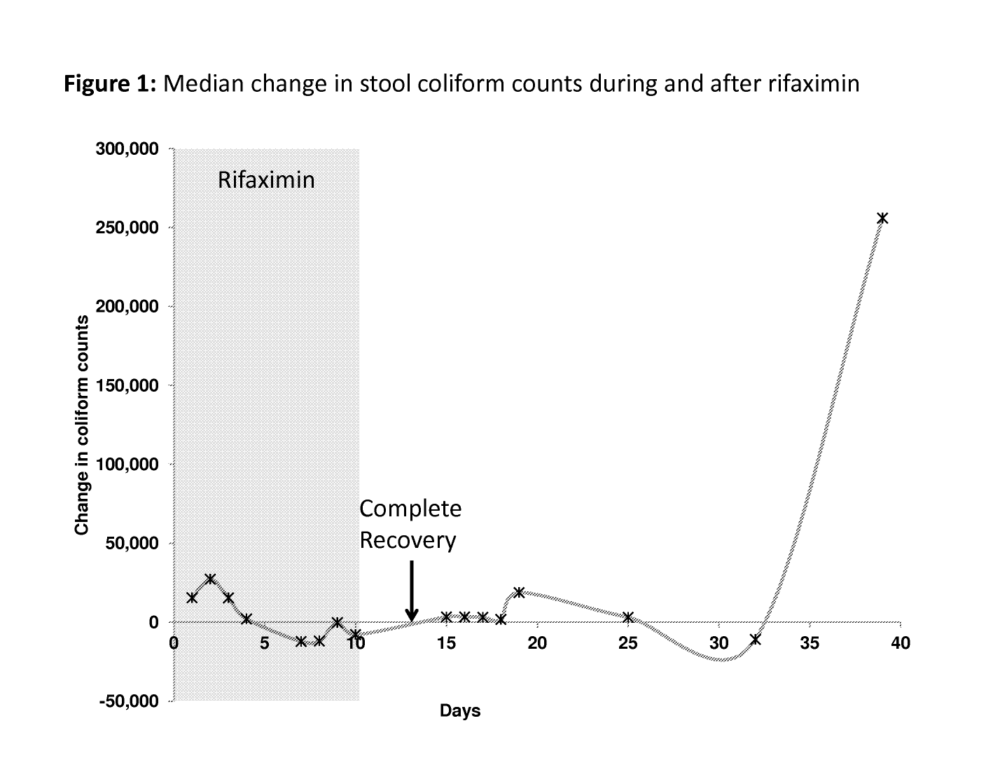

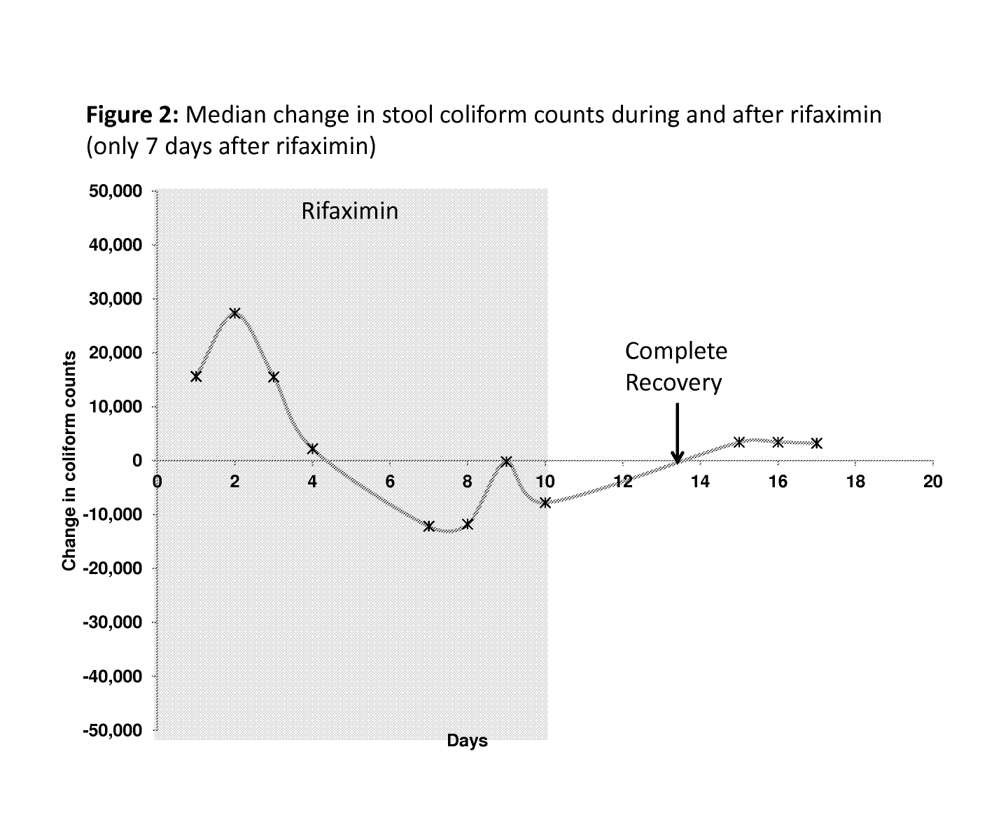

The rats were assessed for effect of rifaximin on stool coliforms (Example 1). A reduction in stool coliforms was seen with rifaximin compared to baseline. At baseline, the rats had a median of 1.86×10⁴ cfu/ml. During the 10-day rifaximin treatment, there was a modest but significant reduction in coliform counts down to 2.2×10³ cfu/mL by day 10 of high dose rifaximin (P<0.01, Wilcoxon Rank sum test for matched pairs). Within three days of rifaximin treatment, the coliform count recovered. FIG. 1 median change in stool coliform counts for up to 30 days after completion of rifaximin treatment, while FIG. 2 shows an expanded view of median change in stool coliform counts for the window of up time up to 7 days after completion of rifaximin treatment.

The results indicate that high dose rifaximin gavage in rats produce modest but significant reductions in coliform counts. However, recovery to normal levels were observed to occur within three days of cessation of therapy (FIG. 1).

### Example 4

### qPCR of the Luminal Bacteria with and without Rifaximin

The same adult male Sprague-Dawley rats from the coliform culture study described above were used here. In this experiment, ten rats were euthanized at day 10 of rifaximin treatment, and ten rats were euthanized at day 30 after completion of rifaximin treatment. As a control to these two groups, a group of 10 rats were gavaged with only PBS (placebo); these rats were also euthanized after 10 days. After euthanasia, rats were dissected to remove a segment of ileum, jejunum and duodenum as well as cecum and left colon. Luminal contents from these 5 areas were removed, and DNA was extracted. Quantitative PCR (qPCR) was used to determine the total number of bacteria in each segment of bowel. These counts were compared between control, rifaximin treated and 30 day post rifaximin rats (Example 1).

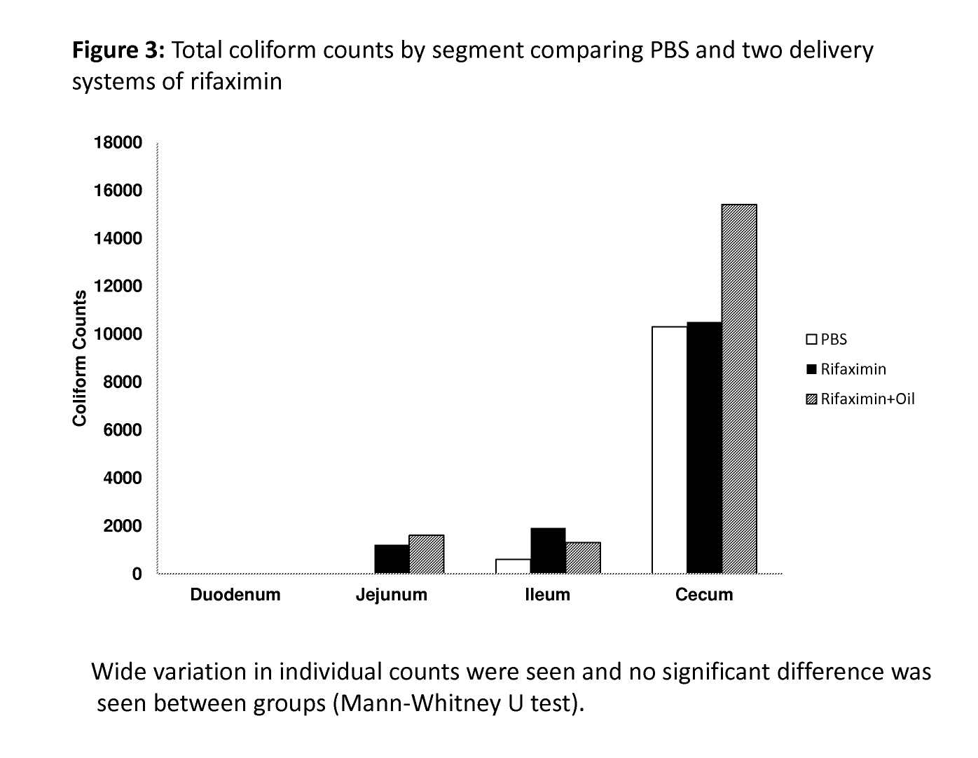

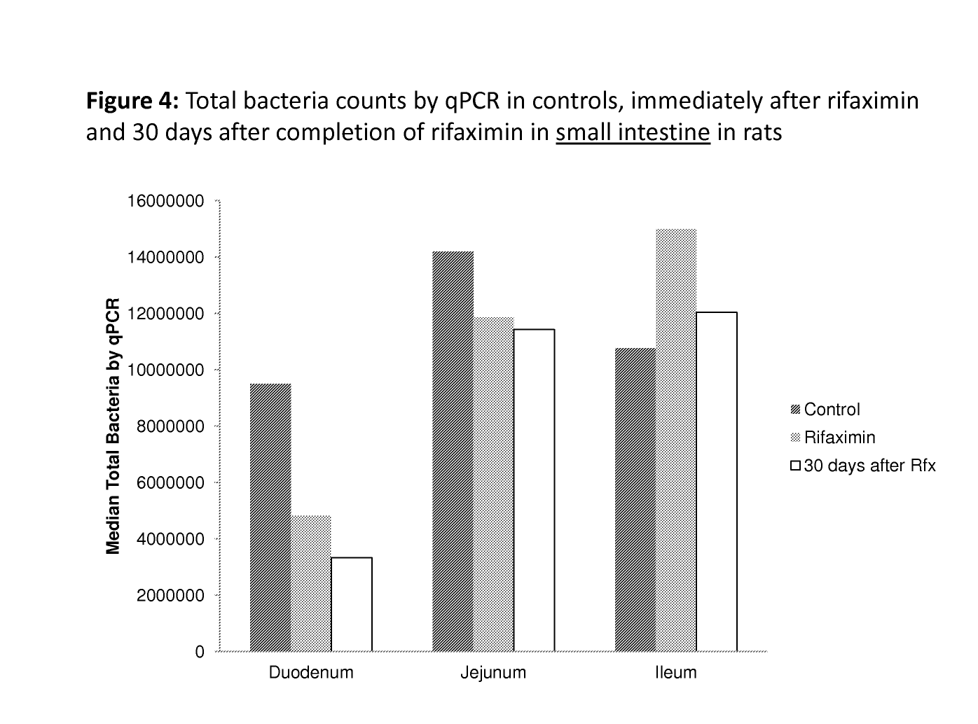

FIGS. 4-7 summarize the findings. Coliform counts in non-treated control rats were seen to increase in number with descent through the bowel with highest counts noted in the cecum and left colon. The duodenum was virtually free of coliforms in these rats similar to humans. After treatment with rifaximin, there was no significant change in coliform counts by culture (FIG. 3).

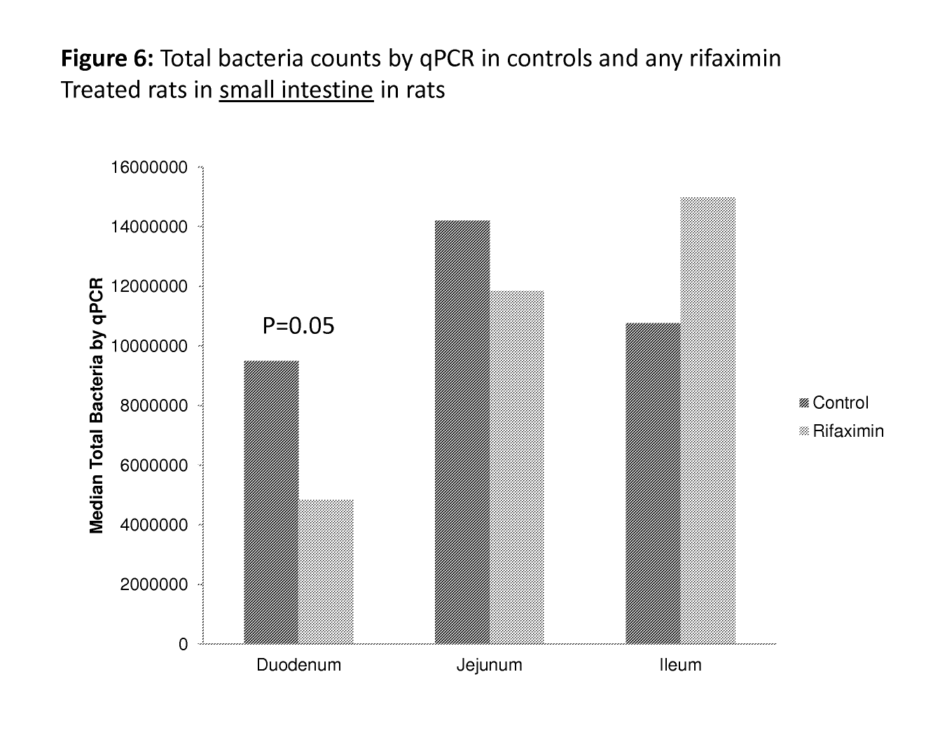

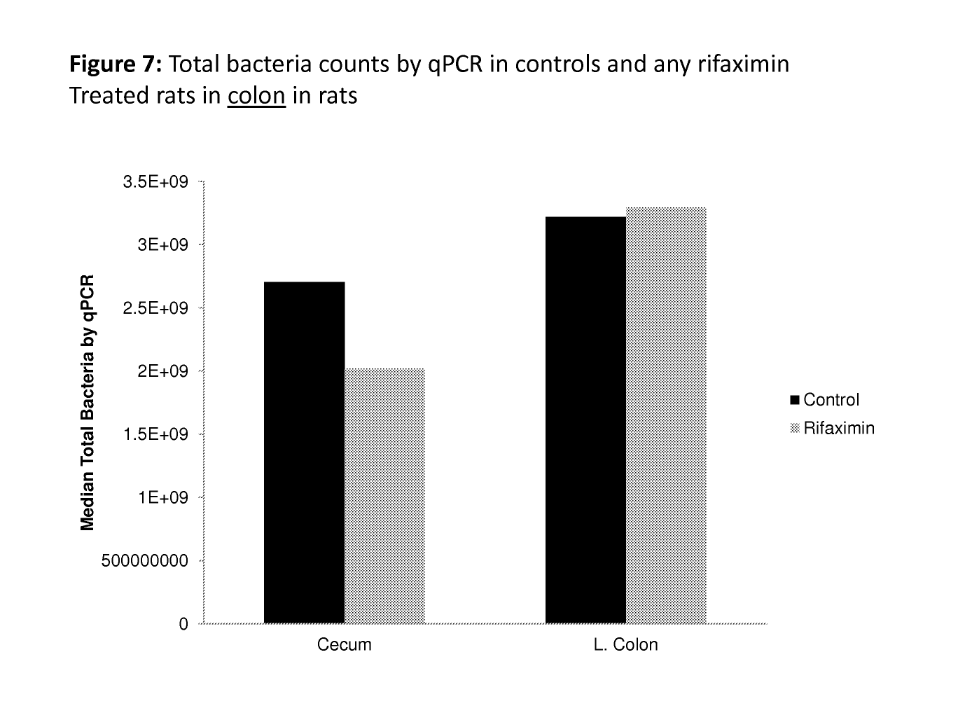

When examining total bacterial counts by qPCR, rifaximin was seen to reduce bacterial levels in the small bowel but not colon. When comparing control rats to all rats receiving rifaximin (n=20) (FIG. 6), there was a significant reduction in duodenal bacterial counts noted. While jejunal counts appeared to diminish as well, this did not reach statistical significance, even at n=20. Beyond the jejunum, no differences were seen in bacterial counts in the ileum, cecum or left colon (FIG. 7).

The data suggest that the effect of rifaximin is predominantly in the duodenum and jejunum with some possible effect in the cecum. However, total left colon counts were unaffected by rifaximin treatment. Interestingly, the reduction in total bacteria by qPCR was seen after acute treatment but continued to decline after completion of rifaximin since counts were even lower by day 30. This suggests the importance of completion of rifaximin but also suggests the change in small bowel total bacteria by rifaximin has a durable effect.

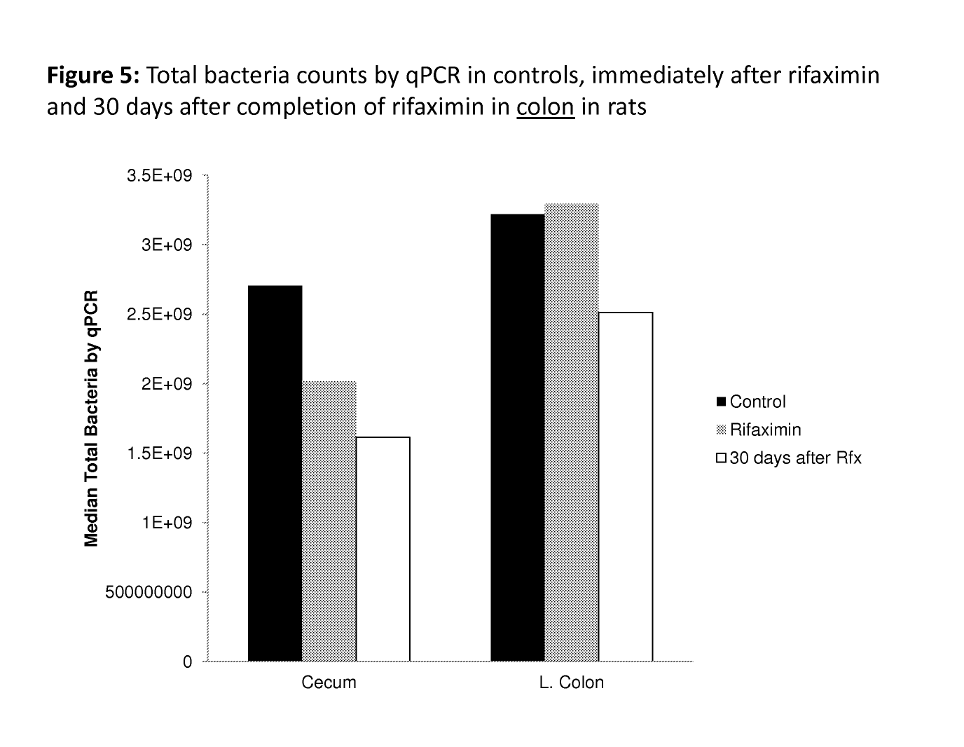

The results indicate that total luminal bacteria in rats treated with rifaximin show the greatest reduction of total bacterial counts in the duodenum and jejunum. Rifaximin appeared to have a durable effect on total duodenal bacterial counts. A reduction in duodenal bacteria counts was observed after 10 days of rifaximin that was sustained 30 days after the completion of rifaximin although this did not quite reach statistical significance (FIG. 4) (P=0.08 by Kruskall-Wallis Test). A similar but non-significant trend was observed in the jejunum. There appeared to be no effect on colonic bacterial counts (FIG. 5).

### Example 5

### Effect of Dietary Fat with Rifaximin on Stool Coliform Bacteria

Adult male Sprague-Dawley Rats were randomly assigned to three groups. Group 1 was gavaged daily for 7 days with rifaximin in PBS. Group 2 received rifaximin 200 mg in lipid daily by gavage. Group 3 were 10 rats only given PBS (control). Rats had fresh stool collected by anal stimulation at baseline and daily during gavage. The stool was homogenized and plated by serial dilution on MacConkey Agar (BD Diagnostics, Franklin Lakes, N.J.). This is a selective agar for coliform bacteria. Based on serial dilution, the total number of coliform bacteria was determined.

A total of 20 male Sprague-Dawley rats were assessed. As expected in healthy animals, few coliforms were seen in the duodenum. The median coliform count in all three groups was 0. However, counts increased into more distal segments of the bowel. Rifaximin did not appear to change normal flora counts in the small bowel of healthy rats either by itself or with oil as a dissolving media.

In conclusion, coliform counts were as expected in control rats with rifaximin having little impact on these counts in healthy rats.

### Example 6

### Effect of Rifaximin on Staphylococcus on Subjects with IBS

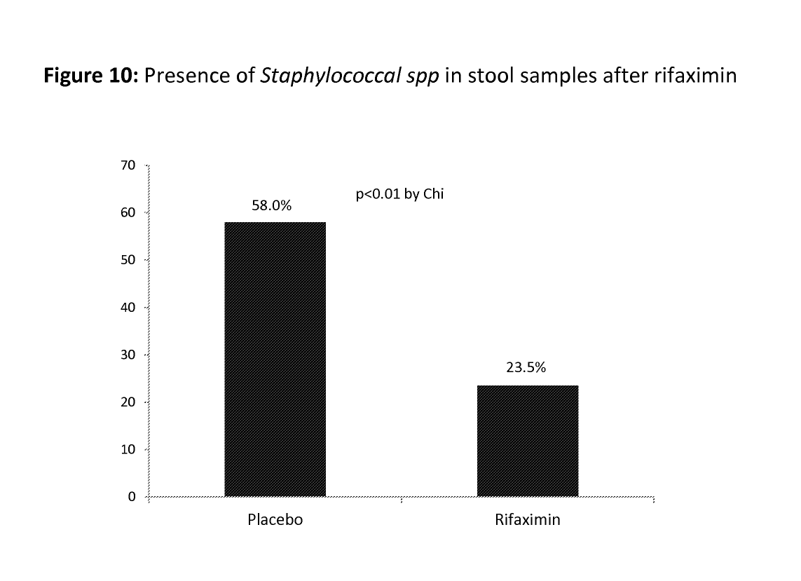

Following acute infection and clearance of C. jejuni, 101 Sprague-Dawley rats exhibiting IBS phenotype were gavaged with placebo and with rifaximin. Fifty rats were gavaged with placebo and 51 rats were gavaged with rifaximin. The rats were then examined for the presence of Staphylococcus. After treatment with rifaximin, there was a reduction in the rate of colonization of rats with Staphylococcus. (FIG. 10)

Among rats with detectable staphylococcus, rifampin resistance was tested using E-test strips and Data was analyzed by Fisher's Exact Test. Results demonstrated that there was no evidence of rifampin resistance after treatment with rifaximin in rats with an IBS phenotype. (Table 2)

TABLE 2
Summary of Staphylococcus sensitivity testing to rifampin.

| | Placebo | Rifaximin | P-value |
|---|---|---|---|
| Median MIC (ug/mL) | 0.016 | 0.214 | 0.45* |
| Range (ug/mL) | 0.008-12 | 0.012-16 | |
| # of sensitive Staphylococcal spp | 28 | 10 | <0.001 |
| # of intermed Staphylococcal spp | 0 | 0 | ND^{¶} |
| # of resistant Staphylococcal spp | 1 | 1 | ND^{¶} |
| % of rats with resistance | 2.0 | 1.96 | ND^{¶} |

*P-value based on Mann-Whitney U non-parametric Test.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the embodiments of the invention as described herein. Such equivalents are intended to be encompassed by the following claims.

What is claimed is:

1. A method of reducing rifampicin-resistance Staphylococcus spp. organisms in a subject in thereof, comprising administering a composition comprising a therapeutically effective amount of rifaximin to the subject.

2. The method of claim 1, wherein reducing results in a reduction in the number of rifampicin-resistant Staphylococcus spp. compared to a pre-treatment baseline number.

3. The method of claim 2, wherein the reduction in the number of rifampicin-resistant Staphylococcus spp. relative to baseline is determined by culturing stool samples obtained from the subject prior and subsequent to administration of the composition.

4. The method of claim 1, wherein the threshold mean inhibitory concentration of rifampicin for the Staphyloccocus spp. organism is less than about 2.5 μg/mL.

5. The method of claim 1, wherein the rifaximin is administered at a dose of about 50 mg to about 6000 mg per day.

6. The method of claim 1, wherein the rifaximin is administered at a dose of between about 100 mg and about 6000 mg; from between about 50 mg and about 2500 mg BID; from between about 50 mg and about 2000 mg TID; 200 mg TID; 200 mg BID or 200 mg QD.

7. The method of claim 1, wherein the rifaximin is administered at a dose of about 550 mg, 600 mg or 1650 mg TID, QD or BID.

8. The method of claim 1, wherein the composition is administered for between about 1 week and about 24 months.

9. The method of claim 1, wherein the composition is administered the composition for about 10 days.

10. The method of claim 1, wherein the subject is suffering from a bowel disease selected from the group of: an inflammatory bowel disease (IBD), hepatic encephalopathy (HE), enteritis, colitis, irritable bowel syndrome (IBS), diarrhea-predominant irritable bowel syndrome (d-IBS), non-constipation-predominant irritable bowel syndrome (non-C IBS), traveler's diarrhea (TD), a Clostridium difficile infection (CDI), diverticular disease, fibromyalgia (FM), chronic fatigue syndrome (CFS), depression, attention deficit/hyperactivity disorder (ADHD), multiple sclerosis (MS), systemic lupus erythematosus (SLE), small intestinal bacterial overgrowth, chronic pancreatitis, and pancreatic insufficiency.

11. The method of claim 10, wherein the inflammatory bowel disease is selected from the group of: Crohn's Disease and ulcerative colitis.

12. The method of claim 10, wherein the enteritis is caused by radiation therapy or chemotherapy.

13. The method of claim 1, further comprising administering a gastrointestinal (GI) cleanser to the subject prior to administration of the composition comprising rifaximin.

14. The method of claim 13, wherein the gastrointestinal cleanser is administered between about 1 to about 90 days prior to administration of the composition.

15. The method of claim 14, wherein the administration of the gastrointestinal cleanser is between about 1 to about 60 days; between about 1 to about 30 days; between about 1 to about 24 days; between about 1 to about 14 days; between about 1 to about 10 days; between about 1 to about 7 days; between about 1 to about 5 days; between about 1 to about 4 days; between about 1 to about 3 days; or between about 1 to about 2 days prior to administration of the composition.
